(19) United States
(12) Patent Application Publication
(10) Pub. No.: US 2023/0184734 A1
(43) Pub. Date: Jun. 15, 2023

(54) METHOD AND APPARATUS FOR MEASURING WATER QUALITY AND USAGE OF TAP WATER

(71) Applicant: GEOGRID Inc., Seoul (KR)

(72) Inventor: Ki Hyun Kim, Namyangju-si (KR)

(21) Appl. No.: 17/581,523

(22) Filed: Jan. 21, 2022

(30) Foreign Application Priority Data
Dec. 9, 2021 (KR) 10-2021-0176030

(51) Int. Cl. G01N 33/18; G01N 21/53; G01N 27/08

(52) U.S. Cl. CPC G01N 33/18; G01N 21/53; G01N 27/08

(57) ABSTRACT

Disclosed are a method and apparatus for automatically measuring water quality and usage of tap water. The method of measuring water quality of tap water includes obtaining, by a control device in the water metering apparatus, water quality data with respect to the large amount of tap water supplied through the distribution pipe using a water quality sensor installed on a particular coupling member among the plurality of coupling members and transmitting, by the control device in the water metering apparatus, the water quality data with respect to the large amount of tap water, which is obtained from the water quality sensor, to an analysis server.

### CROSS-REFERENCE TO RELATED APPLICATION

This application claims priority to and the benefit of Korean Patent Application No. 10-2021-0176030, filed on Dec. 9, 2021, the disclosure of which is incorporated herein by reference in its entirety.

### BACKGROUND

### 1. Field of the Invention

The present invention relates to a method and apparatus for automatically measuring water quality and usage of tap water, and more particularly, to a method of measuring water quality of tap water while a water metering apparatus which receives power from a regular power supply receives a large amount of tap water supplied from a distribution pipe and transfers a certain amount of tap water to each household through a water supply pipe and a plurality of coupling members configured to connect such water supply pipes are present on the distribution pipe. The method includes obtaining, by the water metering apparatus, water quality data with respect to the supplied large amount of tap water using a water quality sensor installed on a particular coupling member among the plurality of coupling members and transmitting, by the water metering apparatus, the water quality data with respect to the large amount of tap water, which is obtained from the water quality sensor, to an analysis server.

### 2. Discussion of Related Art

Generally, rivers, lakes, and the like provide an environment where fish and the like inhabit, and water in rivers, lakes, and the like is purified to be used as drinking water (for example, tap water and the like).

In order to purify the water in rivers, lakes, and the like as described above, the water is purified to be clean through a variety of processes to be drinkable. The purification is executed using a variety of methods such as water intake, chemical treatment, solidification and condensing, precipitation, filtration, disinfection, and the like.

Also, as a water quality sensor configured to measure a contamination level of water, there are used a turbidity sensor configured to measure a turbidity level of water and a glass membrane sensor responding to hydrogen ions. As the glass membrane sensor, there is a pH sensor.

Also, turbidity sensors are configured to optically detect a contamination level of water and classified into a visual turbidity sensor, a transmitted light turbidity sensor, a scattered light turbidity sensor, and an integrating sphere turbidity sensor depending on a measuring method.

In addition, the pH sensor, which is a glass membrane sensor, includes a negative electrode membrane of a thin film formed of special glass which responds to pH of a hemisphere shape on a fore-end of a glass tube called a glass electrode support tube, and an internal liquid of a glass electrode and an internal electrode are present therein. A lead wire extends to the internal electrode, and a fore-end thereof is formed of a terminal for accessing a meter. The pH sensor is widely used.

Generally, until tap water is supplied to a consumer, water is supplied from a water source and tap water is produced through a water purification process and supplied to a consumer through a water supply pipeline via a water reservoir, a booster station, and the like.

Water quality monitoring items for the tap water include eight items, such as phenol, cyan, total organic carbons (TOC), ammonia nitrogen, and the like, in raw water and five items such as pH, turbidity, residual chlorine, electrical conductivity, and a water temperature in a supplying process. Among them, measured values of three items such as pH, turbidity, and residual chlorine which have a close relation with the health of citizens are made public.

Accordingly, for unspecified people who use tap water as drinking water, it is necessary to secure safety in water quality such as preventing a water quality accident by monitoring raw water to a faucet in real time for 24 hours and it is necessary to sense and quickly respond to an abnormal state in advance by operating an early warning system or the like.

Meanwhile, in order to control a water shortage and to remedy environmental problems caused by climate changes, industrialization, and urbanization, a new water integrated administration system, that is, a smart water grid, is necessary. However, it is difficult to apply the smart water grid to a current water meter administration method.

Particularly, since energy is supplied using a battery, it is necessary to periodically replace the battery. Also, in order to maximize the lift of battery, it is necessary to use a low-power wide-area (LPWA) network in which there are limits in transmission speed and data and it is necessary to limit the number of metering times.

This method has a difficulty in metering source data in real time to construct big data. Accordingly, a method of metering real-time tap water usage and constructing big data for efficient water administration so as to remedy social problems is necessary.

Hence, the present applicant intends to provide an apparatus and method for measuring water quality in addition to efficiently remote-metering tap water by coupling a protection box for protecting a water meter with a variety of information and communications technology (ICT) devices (sensors).

### RELATED ART DOCUMENTS

### Patent Documents

Patent Document 0001: Korean Patent Registration No. 10-1621737 (May 11, 2016)

Patent Document 0002: Korean Patent Registration No. 10-2120427 (Jun. 2, 2020)

### SUMMARY OF THE INVENTION

The present invention is directed to providing a method and an apparatus for automatically measuring water quality and usage of tap water in which a water quality sensor is detachably installed on an inlet of a distribution pipe (that is, a position before a plurality of water supply pipes each including a water meter and diverging into respective households to supply tap water thereto are installed) which diverges from a water supply pipeline into a building such as a condominium or apartment in which a plurality of households reside or a particular line of the corresponding building so as to allow a water metering apparatus provided in each building or a particular line of the corresponding building to detect water quality of tap water supplied to the particular building or the particular line and tap water usage of each household in real time. In addition, through a method of automatically transmitting the detected tap water usage and water quality condition information to an analysis server at a remote place through a communication part of the water metering apparatus provided in each building or a particular line of the corresponding building, integrated water quality conditions of tap water supplied to each building or a particular line of the corresponding building, tap water usage for each household, and the like are automatically measured and recognized remotely and big data related to the water quality and usage of tap water are constructed.

The present invention is also directed to providing a method and apparatus for automatically measuring water quality and usage of tap water in which an analysis server is capable of administrating the use of tap water on the basis of stored water quality data by utilizing artificial intelligence (AI).

The present invention is also directed to providing a method and apparatus for automatically measuring water quality and usage of tap water in which an expected problem for each household is remediable on the basis of tap water usage stored in an analysis server.

Other detailed aspects of the present invention will be obviously apparent to and understood by experts or researchers in the art through the following detailed description.

According to an aspect of the present invention, there is provided a method of measuring water quality of tap water while a control device in a water metering apparatus which receives power from a regular power supply receives a large amount of tap water supplied from a distribution pipe and transfers a certain amount of tap water to each household through a water supply pipe and a plurality of coupling members configured to connect such water supply pipes are present on the distribution pipe. The method includes (a) obtaining, by the water metering apparatus, water quality data with respect to the supplied large amount of tap water using a water quality sensor installed on a particular coupling member among the plurality of coupling members and (b) transmitting, by the water metering apparatus, the water quality data with respect to the large amount of tap water, which is obtained from the water quality sensor, to an analysis server.

The distribution pipe may receive the large amount of tap water supplied from a water supply pipeline. Here, the particular coupling member provided on the distribution pipe may include a first particular coupling member, among the plurality of coupling members, which is present at a position closest to the water supply pipeline. Also, the first particular coupling member may be coupled to a first water quality sensor in place of a first particular water supply pipe, and a sensing part of the first water quality sensor may be located inside the distribution pipe and may obtain first water quality data with respect to the large amount of tap water.

While a coupling member, among the plurality of coupling members, which is present at a position closest to the water supply pipeline next to the first particular coupling member, is referred to as a second particular coupling member and the second particular coupling member is included in the particular coupling member, the second particular coupling member may be coupled to a second water quality sensor configured to measure another element different from that of the first water quality sensor in place of a second particular water supply pipe and a sensing part of the second water quality sensor may be located inside the distribution pipe and may obtain second water quality data with respect to the large amount of tap water. Here, the water supply pipes may be connected to the remaining coupling members excluding the first particular coupling member and the second particular coupling member.

The water quality sensor may include at least one of a pH detection sensor configured to measure the activity of hydrogen ions present in tap water, that is, a hydrogen ion concentration quotient (pH), a turbidity detection sensor configured to optically measure a light-scattering degree caused by floating matter by allowing light to be incident onto tap water, a residual chlorine concentration detection sensor configured to measure a concentration of chlorine remaining after disinfection to eliminate pathogenic bacteria in tap water, an electrical conductivity detection sensor, and a water temperature detection sensor.

The water quality data of tap water which is obtained through the water quality sensor may include one or more pieces of data of pH, turbidity, a residual chlorine concentration, electrical conductivity, and a water temperature. Here, the electrical conductivity of tap water may be calculated, at a reference temperature of 25 degrees, using the following equation,

$${C}_{25} = \frac{{C}_{t}}{1 + α ( t - 25 )} . \qquad Equation )$$

Here, C₂₅ may denote an electrical conductivity value at a temperature of 25 degrees, Cₜ may denote an electrical conductivity value at a temperature of t degrees, and a may correspond to a linear temperature coefficient.

While the water supply pipes are connected to the remaining coupling holes of the plurality of coupling members excluding the particular coupling hole and a plurality of water meters are each installed on one of a plurality of water supply pipes, the control device in the water metering apparatus may obtain metering data with respect to certain amounts of tap water transferred to households using the plurality of water meters installed on the other coupling holes and may transmit a plurality of metering data obtained from the plurality of water meters to the analysis server.

While the water metering apparatus may include one communication module and the plurality of water meters including the water quality sensor correspond to the one communication module, the control device in the water metering apparatus may transmit the large amount of water quality data and the plurality of metering data to the analysis server using the one communication module through long distance communication.

While the tap water flowing from the distribution pipe is transferred to each of the plurality of households through the water supply pipe of each household, the control device in the water metering apparatus may measure water quality with respect to a large amount of tap water supplied to a corresponding building or a particular line of the corresponding building for each certain period using the water quality sensor, and the large amount of water quality data measured as described above may be stored in a database included in the analysis server.

A processor included in the analysis server may analyze water quality data with respect to pH, turbidity, a residual chlorine concentration, electrical conductivity, and a water temperature of the large amount of tap water using an AI module and may automatically transmit a result of analyzing to a water supply administrator terminal or a water supply monitoring server which is determined.

As a result of determining, by the processor in the analysis server, water quality with respect to a large amount of tap water supplied to a particular building or a particular line of the corresponding building using the AI module, when at least one piece of data of pH, turbidity, a residual chlorine concentration, electrical conductivity, and a water temperature deviates from a certain range, the analysis server may automatically transmit a warning message which informs a contaminated state of the large amount of tap water supplied to the particular building or the particular line of the corresponding building to the determined water supply administrator terminal or water supply monitoring server, and further, may automatically send a message to stop using tap water to terminals of the corresponding building or each household of the particular line of the corresponding building.

While the tap water flowing from the distribution pipe is transferred to each of the plurality of households through the water supply pipe of each household, the control device in the water metering apparatus may measure daily mean usages of the tap water with respect to the plurality of households using the plurality of water meters and may store the daily mean usages in the database included in the analysis server. When a daily mean tap water usage of a first particular household of the respective households for a particular period deviates from a certain range from a particular daily mean usage, the analysis server may transmit a warning message to a terminal corresponding to the first particular household.

According to another aspect of the present invention, there is provided an apparatus for measuring water quality of tap water while a water metering apparatus which receives power from a regular power supply receives a large amount of tap water supplied from a distribution pipe and transfers a certain amount of tap water to each household through a water supply pipe and a plurality of coupling members configured to connect such water supply pipes are present on the distribution pipe. The apparatus includes a water quality sensor installed on a particular coupling member among the plurality of coupling members and a control device configured to obtain water quality data with respect to the large amount of tap water through the water sensor and to transmit the water quality data with respect to the large amount of tap water, which is obtained from the water quality sensor, to an analysis server.

The water metering apparatus may further include a plurality of water meters installed on the respective water supply pipes to meter and transmit amounts of tap water used in the respective households to the control device.

The water metering apparatus may further include a communication module and may further include an analysis server, at a remote place, which is configured to receive, analyze, and treat water quality data of a large amount of tap water supplied to a particular building or a particular line of the corresponding building and tap water usage data of respective households, which are transmitted through the communication module.

The analysis server may include a cloud server including a communication part and a processor and further include a database.

The analysis server may receive a large amount of water quality data such as pH, turbidity, a residual chlorine concentration, electrical conductivity, and a water temperature from the water metering apparatus on the basis of long distance communication and may store the large amount of water quality data in the database.

A processor included in the analysis server may analyze the large amount of water quality data with respect to pH, turbidity, residual chlorine, electrical conductivity, and a water temperature of the tap water using an AI module and may automatically transmit a result of analyzing to a water supply administrator terminal or a water supply monitoring server which is determined.

The processor in the analysis server may analyze a tap water usage of each household using the AI module, and when a particular household uses an excessively larger or smaller amount than usual, may automatically transmit the tap water usage to a particular user terminal designated by the corresponding household.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a schematic diagram of an entire system which measures water quality and usage according to one embodiment of the present invention;

FIG. 2 is a flowchart illustrating a process of transmitting a large amount of water quality data to an analysis server according to one embodiment of the present invention; and

FIG. 3 is a detailed configuration diagram of a water metering apparatus according to one embodiment of the present invention.

### DETAILED DESCRIPTION OF EXEMPLARY EMBODIMENTS

The following detailed description of the present invention refers to the attached drawings which illustrate a particular embodiment as an example in which the present invention is implementable. This embodiment will be described in detail to allow those skilled in the art to adequately implement the present invention. It should be understood that a variety of embodiments of the present invention differ from one another but are not mutually exclusive. For example, a particular shape, structure, and feature disclosed herein will be implemented, in conjunction with one embodiment, as another embodiment without departing from the concept and scope of the present invention. Also, it should be understood that positions and arrangement of individual elements in each disclosed embodiment may vary without departing from the concept and scope of the present invention. Accordingly, it should be noted that the following detailed description is not provided to have a limitative meaning, and the scope of the present invention, when appropriately described, will be defined only by the attached claims in addition to all equivalents of the claims. In the drawings, like reference numerals refer to like or similar functions throughout a variety of aspects.

Hereinafter, in order to allow one of ordinary skill in the art to easily implement the present invention, exemplary embodiments of the present invention will be described in detail with reference to the attached drawings.

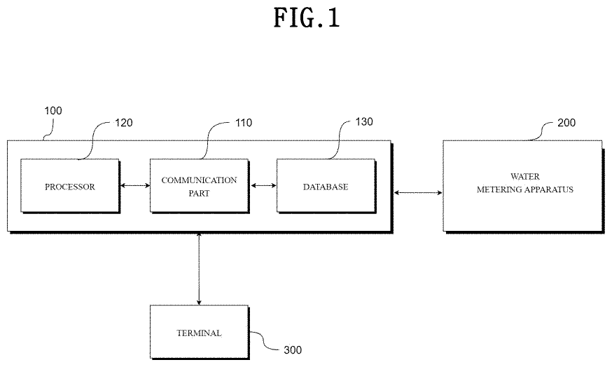

FIG. 1 is a schematic diagram of an entire system which measures water quality and usage according to one embodiment of the present invention.

As shown in FIG. 1, the entire system according to the present invention may, roughly, include an analysis server 100 and a water metering apparatus 200 including a water quality sensor 280 and the like. Also, in some cases, as shown in FIG. 1, the entire system may include a plurality of user terminals 300, a water supply administrator terminal, a water supply monitoring server, and the like.

First, the analysis server 100 according to the present invention includes a communication part 110 and a processor 120, and in some cases, may not include a database 130 unlike FIG. 1. For reference, the analysis server 100 may correspond to a certain cloud server so as to perform communication with the water metering apparatus or the like.

First, the analysis server 100 may transmit and receive information with the water metering apparatus 200 or the like through the communication part 110, and the communication part 110 of the analysis server 100 may be implemented using a variety of communication techniques. That is, technology such as Wi-Fi, wideband code division multiple access (WCDMA), high speed downlink packet access (HSDPA), high speed uplink packet access (HSUPA), high speed packet access (HSPA), mobile WiMAX, WiBro, long term evolution (LTE), 5G, Bluetooth, infrared data association (IrDA), near field communication (NFC), ZigBee, wireless local area network (LAN), and the like may be applied. Also, when services are provided over the Internet, the services may follow a transmission control protocol/Internet protocol (TCP/IP) which is a standard protocol for transmission of information over the Internet.

Subsequently, the database 130 according to the present invention may store data related to tap water with respect to each of a plurality of households. When an external database is used, the analysis server 100 may access the external database through the communication part 110.

Also, the analysis server 100 may perform communication with the water metering apparatus 200, the terminals 300, and the like through the communication part 110.

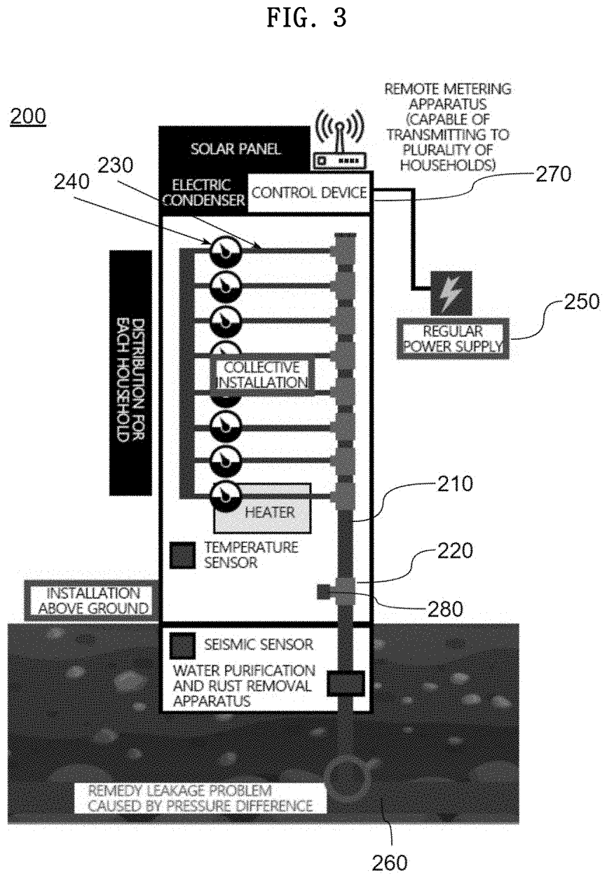

The water metering apparatus 200 may include a plurality of measuring sensors (for example, a temperature sensor, a water pressure sensor, a leakage sensor, a vibration sensor, a seismic sensor, an electric conductivity measuring sensor, a turbidity measuring sensor, a pH measuring sensor, and the like) and may be connected to a distribution pipe 210 configured to transfer tap water. Tap water flowing from the distribution pipe 210 may be transferred to a plurality of households through the water metering apparatus 200. The water metering apparatus 200 may check the tap water transferred to the plurality of households using the plurality of measuring sensors. The water metering apparatus 200 will be described in detail with reference to FIG. 3.

Also, in the case of the terminal 300, any digital devices configured to perform communication, such as a memory device, and a microprocessor to be equipped with operation ability such as a desktop computer, a laptop computer, workstation, a personal digital assistant (PDA), a web pad, a mobile phone, a smart remote controller, a variety of Internet of Things (IoT) main devices, or the like, may correspond to the terminal 300 according to the present invention. The terminal 300 may correspond to a terminal of a user who receives tap water or a terminal of an interested person related to the corresponding user.

That is, the processor 120 of the analysis server 100 may transmit a necessary message to the user or the interested person through the terminal 300.

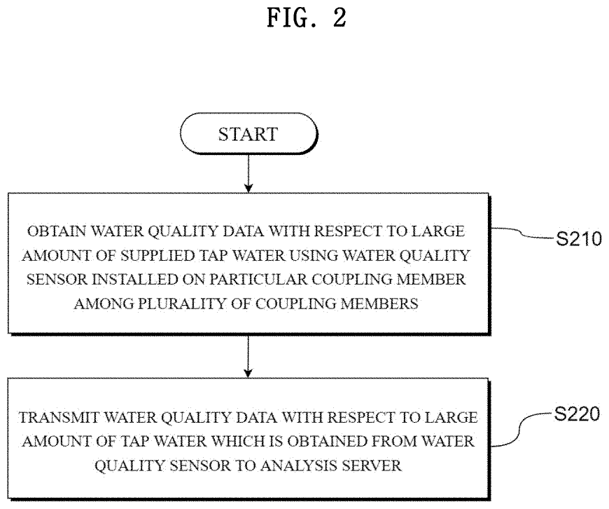

FIG. 2 is a flowchart illustrating a process of transmitting a large amount of water quality data to an analysis server according to one embodiment of the present invention. FIG. 3 is a detailed configuration diagram of the water metering apparatus according to one embodiment of the present invention.

As shown in FIG. 3, the water metering apparatus 200 may receive power through a regular power supply 250 and may receive a large amount of tap water supplied from the distribution pipe 210.

Also, the water metering apparatus 200 may transfer a certain amount of tap water of the large amount of supplied tap water to each household through a water supply pipe 230, and a plurality of coupling members 220 configured to connect the water supply pipe 230 may be present on the distribution pipe 210.

The water metering apparatus 200 may be installed on each distribution pipe 210 which diverges from a water supply pipeline 260 to a particular building such as condominiums, apartment building, and the like in which a plurality of households reside or a particular line of the corresponding building. The distribution pipe 210 may be configured to receive a large amount of tap water from the water supply pipeline 260 and to transfer a certain amount of tap water to a plurality of households through a plurality of water supply pipes 230.

Also, the plurality of coupling members 220 may be present on one side of the distribution pipe 210 which extends into the water metering apparatus 200 to install and connect the water supply pipe 230, which diverges to each household, the water quality sensor 280, and the like.

Here, the coupling members 220 may have a variety of shapes such as a T-shape, Y-shape, and the like and include an elbow, connecting hole, and the like. In some cases, unlike that shown in FIG. 3, an opening-shaped connection hole which is connectable to the water supply pipe 230 or the like may be present. Here, the connection hole may also be included in the coupling member 220. Eventually, the coupling member 220 may have a variety of shapes and may include a function of connecting the water supply pipe 230 or the water quality sensor 280 to the distribution pipe 210.

For reference, when the water supply pipe 230 or the water quality sensor 280 is not coupled to any one of the coupling members 220 provided on the distribution pipe 210, a pipe end having a cap or nut shape to prevent tap water leakage may be detachably coupled to an outlet of the coupling member 220.

Also, the water metering apparatus 200 may obtain water quality data with respect to a large amount of tap water supplied through the water quality sensor 280 installed on a particular coupling member of the plurality of coupling members 220 (S210).

Generally, the plurality of water supply pipes 230 may be connected to the distribution pipe 210 of the water metering apparatus 200 so as to transfer tap water to each household. In the present invention, any one of the plurality of water supply pipes 230 may be eliminated from the distribution pipe 210, and the water quality sensor 280 may be coupled instead thereof. There is an effect of coupling the distribution pipe 210 to the water quality sensor 280 using the existing coupling member connected to the water supply pipe 230 without additional installation.

Although the particular coupling member may correspond to any one coupling member of the plurality of coupling members 220, a so-called “first particular coupling member” which is a coupling member 220-1 present at a position closest to the water supply pipeline 260 among the plurality of coupling members 220 may be included in the particular coupling member.

The first particular coupling member 220-1 may be coupled to a first water quality sensor in place of a first particular water supply pipe. That is, the first particular coupling member 220-1 may replace the first particular water supply pipe present at the position closest to the water supply pipeline 260 among the plurality of water supply pipes 230 and may be coupled to the first water quality sensor.

Here, a sensing part of the first water quality sensor may be located inside the distribution pipe 210 and obtain first water quality data with respect to the large amount of tap water.

That is, when any one household of a plurality of households uses tap water while the water quality sensor 280 is detachably installed on the first particular coupling member 220 present at the position closest to the water supply pipeline 260 among the plurality of coupling members 220 provided on the distribution pipe 210 diverging from the water supply pipeline 260 in the water metering apparatus 200 provided on each building or a particular line of the corresponding building, before distributing and supplying the tap water to each household, water quality data with respect to a large amount of tap water flowing into the distribution pipe 210 may be detected at an entrance of the distribution pipe 210.

Also, a coupling member present at a position closest to the water supply pipeline 260 next to the first particular coupling member 220-1 among the plurality of coupling members 220 may be set to be a second particular coupling member 220-2 and to be included in the particular coupling member.

The second particular coupling member 220-2 may be coupled to a second water quality sensor in place of a second particular water supply pipe. That is, the second particular coupling member 220-2 may replace the second particular water supply pipe present at the position secondarily closest to the water supply pipeline 260 among the plurality of water supply pipes 230 and may be coupled to the second water quality sensor.

Also, a sensing part of the second water supply sensor may be located inside the distribution pipe 210 and obtain second water quality data with respect to the large amount of tap water, and the second water quality sensor may measure a factor different from that of the first water quality sensor.

In detail, the water quality sensor including the first water quality sensor and the second water quality sensor which have been described above may correspond to at least one of a pH detection sensor configured to measure the activity of hydrogen ions present in tap water (that is, a hydrogen ion concentration quotient (pH)), a turbidity detection sensor configured to optically measure a light-scattering degree caused by floating matter by allowing light to be incident onto tap water, a residual chlorine concentration detection sensor configured to measure (measurable from 0 to 10 ppm) a concentration of chlorine remaining after disinfection to eliminate pathogenic bacteria in tap water, an electrical conductivity detection sensor, and a water temperature detection sensor.

Since the first water quality sensor and the second water quality sensor which have been described above measure different factors, when the first water quality sensor is a pH detection sensor, the second water quality sensor may correspond to any one of other sensors excluding the pH detection sensor.

Among the sensors, the electrical conductivity detection sensor indicates CTD which is short for conductivity, temperature, and depth. In addition, general CTD equipment including the electrical conductivity detection sensor is an apparatus capable of measuring a water temperature and salinity at the same time for each water level on the spot.

Therefore, when the electrical conductivity detection sensor is selected as the water quality sensor 280 in the present invention, without additionally installing a residual chlorine concentration detection sensor and a temperature detection sensor, a concentration of chlorine contained in tap water and a water temperature may be detected. Also, a temperature, electrical conductivity, and a water pressure may be electrically measured so as to calculate salinity from the electrical conductivity and water pressure excluding a water level because of being installed on the distribution pipe.

Here, when CTD is used as the water quality sensor 280, regular calibration with respect to the sensor is necessary. Also, depending on purpose of investigation, in addition to the electrical conductivity detection sensor, a variety of detection sensors configured to detect dissolved oxygen (DO), pH, light transmission, and the like may also be further detachably mounted on the entrance of the distribution pipe 210 to be used.

Additionally, as the water quality sensor 280 may include, in addition to the above detection sensors, a water pressure detection sensor, a leakage detection sensor, a vibration detection sensor, a seismic tremor detection sensor, and the like may be further installed and used.

Among the plurality of coupling members 220, excluding the particular coupling members, that is, the first particular coupling member and the second particular coupling member, the water supply pipes 230 may be connected to the remaining coupling members 220-3, 220-4, and . . . , and a plurality of water meters 240 may each be installed on one of the plurality of water supply pipes 230. That is, the water meter 240 configured to meter an amount of certain tap water supplied to a household is installed on each of the water supply pipes 230.

The water metering apparatus 200 may obtain metering data with respect to respective amounts of certain tap water transferred to households using a plurality of water meters each installed in one of the remaining coupling members 220-3, 220-4, and . . . . Also, a plurality of such metering data obtained from the plurality of water meters may be transferred to the analysis server 100.

Likewise, the water metering apparatus 200 may also transfer water quality data with respect to the large amount of tap water which is obtained from the water quality sensor 280 to the analysis server 100 (S220).

Meanwhile, the analysis server 100 may receive, on the basis of long distance communication through the communication part 110, a large amount of integrated water quality data such as pH, turbidity, residual chlorine, electrical conductivity, and a water temperature which are obtained through the water quality sensor 280 and tap water usage data of respective households which are obtained through the water meters 240 of respective households through the water metering apparatus 200 and may store the water quality data and tap water usage data in the database 130.

Also, the processor 120 in the analysis server 100 may analyze water quality data such as pH, turbidity, residual chlorine, electrical conductivity, and a water temperature of the tap water using an artificial intelligence (AI) module provided in the processor 120 and may automatically transfer a result of analysis to a predetermined water supply administrator terminal 500 or a water supply monitoring server 600 which is determined. Also, as necessary, a water quality analyzing result and a necessary message may be transferred to a particular user terminal designated by each household in a particular building or a particular line of the corresponding building.

Also, the processor 120 in the analysis server 100 may analyze tap water usage in each household using the AI module provided therein, and when a particular household uses an excessively larger amount of tap water or an excessively smaller amount of tap water than usual, a message corresponding thereto may be automatically transferred to a particular user terminal 400 designated by the corresponding household.

In an existing water metering system, it is necessary to periodically replace a battery and there is a difficulty in real-time inspection (for inundation, leakage, sanitary problems, and metering) because the system itself is embedded in the ground. However, in the case of the water metering apparatus 200 according to the present invention, a freezing and bursting problem is remedied by maintaining an internal temperature above zero so that the water metering apparatus 200 is installable above the ground.

Additionally, the water metering apparatus 200 used in the present invention may receive necessary power (energy) supplied from the regular power supply 250 and additionally receive energy (power) from a solar panel.

Since the power is supplied from the regular power supply 250 and the solar panel and the like as described above, in the present invention, a variety of types of data may be measured in real time or according to a certain cycle using a variety of devices such as an integrated circuit temperature (ICT) sensor and the like and related big data may be constructed. For reference, in the present invention, inspections may be performed per minute using sensors and the like and about 1,440 measurements may be performed per day.

Meanwhile, since an existing water quality sensor, like the water meters 240 of respective households, is configured to be installed on each of the plurality of water supply pipes 230 diverging from the distribution pipe 210 into the respective households through the plurality of coupling members 220 to supply tap water thereto, although water quality with respect to a large amount of tap water actually supplied through the distribution pipe 210 is approximately similar to water quality of tap water supplied while being distributed through the water supply pipes 230 of the respective households, an unnecessarily great number of water quality sensors 280 are installed and thus costs increase.

Particularly, since a system configured to integrate, collect, and transfer respective pieces of water quality information detected from the water quality sensors 280 to a water supply administration server or the like in real time is not provided, there is a great difficulty in automatically collecting water quality of tap water supplied to each building or each household of a particular line of the corresponding building and taking immediate corresponding action when an abnormal state occurs.

However, the water quality sensor 280 applied to the present invention is detachably installed on the coupling member 220 located to be closest to the water supply pipeline 260 among the plurality of coupling members 220 provided on the distribution pipe 210 connected to the water supply pipeline 260 in the water metering apparatus 200 provided on each building or a particular line of the corresponding building as shown in FIG. 3 so as to detect and directly transfer overall water quality condition information with respect to a large amount of tap water supplied to the corresponding building or the particular line of the corresponding building to a control device 270 provided in the water metering apparatus 200. Also, the water metering apparatus 200 always maintains an internal temperature above zero so as to remedy a freezing and bursting problem so that the water metering apparatus 200 is installable above the ground.

Meanwhile, the analysis server 100 may allow the control device 270 in the water metering apparatus 200 to obtain integrated water quality data with respect to a large amount of tap water in real time using the water quality sensor 280.

Here, as described above, the water quality sensor 280 included in the water metering apparatus 200 may include one or more detection sensors among a pH detection sensor, a turbidity detection sensor, a residual chlorine concentration detection sensor, an electrical conductivity detection sensor, and a water temperature detection sensor. Also, the water quality data may include one or more pieces of data with respect to pH, turbidity, a residual chlorine concentration, electrical conductivity, and a water temperature.

Here, the electrical conductivity detection sensor included in the water quality sensor 280 may measure salts in ionic status included in tap water by measuring electrical conductivity so as to detect water quality in real time.

Also, in the case of the electrical conductivity detection sensor, a change in temperature value may have a great influence on a measured value when electrical conductivity is measured. Accordingly, a measuring device corrects a temperature and may have high accuracy in controlling chemical concentrations by adjusting a gradient of 0 to 5%.

In detail, in the electrical conductivity detection sensor, a reference temperature may be 25 degrees and electrical conductivity of tap water may be calculated according to the following Equation.

$${C}_{25} = \frac{{C}_{t}}{1 + α ( t - 25 )} \qquad Equation )$$

Here, C₂₅ may denote an electrical conductivity value at a temperature of 25 degrees, Cₜ may denote an electrical conductivity value at a temperature of t degrees, and a may correspond to a linear temperature coefficient. Here, a may be selected within a range of 0 to 5% per ° C., generally about 2% per ° C. In general, in the case of acid, the linear temperature coefficient may be smaller (for example, 1.6%). In the case of base, the linear temperature coefficient may be greater (for example, 2.2%).

When an electrical conductivity value (on the basis of 25 degrees) measured by the electrical conductivity detection sensor is greater than or equal to a contamination reference value, the processor 120 in the analysis server 100 may automatically notify the water supply administrator terminal which supervises supplying of the tap water or the water supply monitoring server of status information and may send a warning message to the user terminal 300 designated by a particular building or each household in a particular line of the corresponding building to ban the use.

For reference, the contamination reference value may vary according to a destination to which tap water is transferred. In detail, in places such as a school, hospital, and the like where a risk of contamination is higher, the contamination reference value may be higher than in other places (for example, a factory).

Also, the water metering apparatus 200 applied to the present invention may include one communication part or communication module, and the water quality sensor 280 and the plurality of water meters 240 may correspond to the one communication module.

Also, the control device 270 in the water metering apparatus 200 may transmit the water quality detection data and the plurality of metering data with respect to a large amount of tap water supplied to a particular building or a particular line of the corresponding building to the analysis server 100 through long distance communication using the one communication module. That is, a one-to-many transmission method is used. For reference, the communication module included in the water metering apparatus 200 may include a sort of transmitter or receiver.

Also, the transmitter of the water metering apparatus 200 may transmit data (water quality condition detection data, tap water usage metering data of each household, and the like) collected using an RS-485 (serial communication) method to the analysis server 100. Here, the RS-485 (serial communication) method may be a communication method of sequentially transmitting or receiving data bit by bit through a transmission line.

According to one embodiment, unless separate data is transmitted from the analysis server 100, the communication module of the water metering apparatus 200 may transmit the collected data such as water quality conditions of tap water and tap water usage of each household to the analysis server 100 using the RS-485 method. That is, the control device 270 in the water metering apparatus 200 and the analysis server 100 may not transmit or receive data at the same time, and thus, when one side thereof transmits data, the other side may only receive data.

Also, the control device 270 in the water metering apparatus 200 may receive information from the analysis server 100 and may transmit data to the analysis server 100 after a certain time (standby time (timeout)) passes. This is to prevent a loss in information caused by transmitting information from both sides at the same time.

The analysis server 100 according to the present invention may receive information from a plurality of such water metering apparatuses 200 and perform administration. Each of the plurality of water metering apparatuses 200 may be installed in one of buildings at different positions or a particular line of the corresponding building so as to measure conditions of tap water supplied to the corresponding building or the particular line of the corresponding building and simultaneously to meter tap water usage or the like with respect to several households in the corresponding building or the particular line of the corresponding building.

Also, as described above, the control device 270 in the plurality of water metering apparatuses 200 may transmit or receive information using the RS-485 communication method. In detail, the analysis server 100 is a certain type of main device and may transmit an instruction or the like to each of the plurality of water metering apparatuses 200 corresponding to sub devices.

Also, according to one embodiment of the present invention, the water metering apparatus 200 may collect information with respect to the plurality of water meters 240 and the plurality of sensors 280 using the RS-485 communication method and may perform communications with the analysis server 100 through one communication module. Here, communications between the communication module of the water metering apparatus 200 may be performed using any one of a variety of communication methods such as IoT, WCDMA, HSDPA, HSUPA, HSPA, mobile WiMAX, WiBro, LTE, 5G, Bluetooth, IrDA, NFC, ZigBee, wireless LAN, and the like.

For reference, any one of the plurality of water meters 240 and the plurality of sensors which are included in the water metering apparatus 200 may transmit, as a certain type of main device, an instruction with respect to each of others corresponding to sub devices.

Each of the plurality of water metering apparatuses 200 may perform communications with the analysis server 100 using the one communication module. The analysis server 100 and the plurality of water metering apparatuses 200 may perform communications using a common communication line (for example, a two-wire method and a four-wire method), and additionally, may perform wireless communications through a network.

Here, the analysis server 100 (main device) may provide the plurality of water metering apparatuses 200 (sub devices) with the authority to use a communication line or a particular wireless communication channel.

The analysis server 100 which is the main device may regularly transmit instructions (for example, to transmit collected data with respect to water quality, usage for each household, and the like of tap water) to each of the sub devices (water metering apparatuses). A detailed description related thereto will be set forth below. For convenience of description, hereafter, the analysis server 100 will be set to be a main device and the water metering apparatus 200 will be set to be a sub device.

To easily perform communications, in the present invention, each of the sub devices may be designated with a unique identification number. Through the unique identification number, the main device may transmit a particular instruction to a particular sub device (here, an identification number is 0xfff).

However, it may be necessary to see whether an error occurs in any one of a plurality of such sub devices. In this case, the main device may sequentially transmit request messages (for example, to respond on whether a signal is satisfactory and to transmit collected data) to the plurality of sub devices, may receive response messages (for example, a response message in which a signal is satisfactory and a message including collected data) from the plurality of sub devices, and when the response message is not transmitted, may see that an error occurs in the corresponding sub device). Here, the main device may wait for the response message from the sub device while standing by for a certain time.

The standby time (timeout) may vary according to time of receiving the collected data (metering data and the like) and may be greater than the time of receiving the collected data. This is because the main device may receive the collected data first and then receive the response message. That is, although the response message is not received while the collected data is received, it may not be determined that an error occurs in the corresponding sub device.

Since the water metering apparatus 200 that is the sub device performs, at the same time, detecting water quality of tap water supplied to a corresponding building or a particular line of the corresponding building and metering usage of tap water used in each of a plurality of households, it is necessary that the standby time with respect to the water metering apparatus 200 is more than transmission time of collected data of a household which uses a largest amount of tap water among the plurality of households.

Since each of the plurality of water metering apparatuses 200 obtains water quality data on water quality of tap water supplied to each of different buildings or a particular line of the corresponding building through the water quality sensor 280 and performs metering with respect to different households, in some cases, respective standby times with respect to the plurality of water metering apparatuses 200 may differ from each other.

However, a plurality of such standby times with respect to the plurality of water metering apparatuses 200 may all be equal which are more than the transmission time of the collected data of the particular household which uses the largest amount of tap water among all households.

Also, when a response message is not received from a particular sub device, the main device may repeatedly transmit a request message a preset number of times. Here, the preset number of times may vary according to the number of the plurality of sub devices, the standby times, and a time limit and will be described with reference to the following equation.

number of sub devices×standby time×preset number of times≤time limit Equation)

For convenience of description, as an example, the time limit may be set to be 15 seconds, the number of sub devices may be set to be 10, and the standby time may be set to be 500 msec. Here, since it is necessary that 10×500 m/sec×preset number of times is smaller than or equal to 15, the preset number of times may correspond to three or less. That is, the main device may repeatedly transmit request messages one to three times.

However, data may be transmitted using another method and may be transmitted using a variety of techniques such as LTE, Wi-Fi, and the like. In some cases, the water metering apparatus 200 may include one or more transmitters and receivers (communication modules).

Also, as shown in FIG. 3, the water metering apparatus 200 may include the plurality of water meters 240 installed on the water supply pipes 230 connected to respective households. The tap water diverging and flowing from the distribution pipe 210 through the coupling member 220 may be transferred to each of the plurality of households through each of the water supply pipes 230.

Here, each of the plurality of water meters 240 may see metering data of tap water (for example, tap water usage and the like) which flows into each of the plurality of households.

Accordingly, the database 130 may store a daily mean tap water usage measured with respect to each of the plurality of households (for example, household a, household b, and the like) using the plurality of water meters 240.

Additionally, the database 130 may also store household information (for example, the ages of household members and the number of household members) with respect to each of the plurality of households. For example, there is stored information that the household a includes one male and the household b includes four members such as a male in his fifties, a female in her fifties, a male in his twenties, and a female in her teens.

While the daily mean tap water usage is stored in the database 130, it may be assumed that a daily mean tap water usage of a first particular household for a certain period (for example, one week) deviates from a certain range from a particular daily mean tap water usage (for example, 300 L) with respect to the first particular household.

Here, the processor 120 of the analysis server 100 may transmit a warning message, which corresponds to the first particular household, that tap water usage is excessively higher or lower than usual to a preset user terminal 400. The above process may be performed using the AI module included in the processor 120 of the analysis server 100.

Here, the user terminal 400 corresponding to the first particular household may correspond to a terminal prestored in the database 130. For example, when the first particular household includes a single senior citizen a in his or her seventies, the terminal may correspond to a terminal of the single senior citizen a or a terminal of a child a′ of the single senior citizen (alternatively, a terminal of a related department such as a single senior citizen protection group and the like).

When tap water usage of the first particular household a is excessively lower than usual, the child a′ may directly check what has happened to the health of the single senior citizen a and may take measures.

On the other hand, when a daily mean tap water usage of one household deviates from a certain range while a number of members of the household is recorded as four or more in the database 130, the processor 120 of the analysis server 100 may not transmit an abnormality-warning message unless a separate request is made from the corresponding household. That is, it may be determined depending on the number of members in a household whether to transmit the warning message.

Also, when the number of members in a household is large, a certain range (based on a daily mean usage) that is a reference for transmitting the warning message may be larger than that when the number of members in a household is small. This is because, when the number of members is large, a tap water usage range (from small usage to large usage) may be large.

Also, the processor 120 of the analysis server 100 may allow or support a tap water usage cost of the second particular household having a smallest daily mean usage among a plurality of daily mean usages with respect to the plurality of households to be charged with a discounted value. That is, the analysis server 100 may directly charge a tap water usage cost or may support another system (for example, a water supply and drainage service and the like) to charge a tap water usage cost.

Here, a discount rate of the discounted value may be determined on the basis of a mean value A with respect to a daily mean usage of the plurality of households and a daily mean usage B of the second particular household. In detail, it may be satisfied that discount rate=A−B/A.

For example, when the mean value A of daily mean usages of the plurality of households is 300 L and the daily mean usage B of the second particular household is 200 L, the discount rate may be obtained as 300-200/300=33.333%, that is, about 33%.

In some cases, the processor 120 of the analysis server 100 may provide the user terminal 400 of the second particular household with a mini game and may determine the discount rate according to a result of the mini game (discount by 33%, discount by 50% or no discount). This is to allow a server (application) of the present invention to be used while reducing unnecessary tap water usage.

Also, according to another embodiment of the present invention, a plurality of households may be divided into two or more groups and a household having a smallest daily mean usage may be selected for each group.

Also, the processor 120 of the analysis server 100 may provide each of the user terminals 400 of members of the selected household with a mini game and may determine so that different discount rates (discount by 33%, discount by 50%, or no discount) may be provided according to a result of the mini game.

The above embodiments of the present invention may be implemented as the form of a program instruction executable through a variety of computer elements and recorded on a computer-readable recording medium. The computer-readable recording medium may include each or a combination of a program instruction, a data file, a data structure, and the like. Program instructions recorded on the computer-readable recording medium may be particularly designed for the present invention or may be well known to those skilled in the field of computer software to be usable. For example, the computer-readable recording medium includes a hard disk, a floppy disk, a magnetic medium such as a magnetic tape, an optical medium such as a compact disc read-only memory (CD-ROM) and a digital versatile disc (DVD), a magneto-optical medium such as a floptical disk, and a hardware device such as a read-only memory (ROM), a random-access memory (RAM), a flash memory, and the like which are particularly configured to store and execute program instructions. For example, the program instruction includes a machine language code made by a compiler, a high-level language code executable by a computer using an interpreter, or the like. The hardware device may be configured to operate through one or more software modules to execute treatment according to the present invention, and vice versa.

According to the present invention, a method and an apparatus for automatically measuring water quality and usage of tap water may provide the following effects. First, a water quality sensor is detachably installed on an inlet of a distribution pipe (that is, a position before a plurality of water supply pipes each including a water meter and diverging into respective households to supply tap water thereto are installed) which diverges from a water supply pipeline into a building such as a condominium or apartment in which a plurality of households reside or a particular line of the corresponding building so as to allow a water metering apparatus provided in each building or a particular line of the corresponding building to detect water quality of tap water supplied to the particular building or the particular line of the corresponding building and tap water usage of each household in real time. In addition, through a method of automatically transmitting the detected tap water usage and water quality condition information to an analysis server at a remote place through a communication part of the water metering apparatus, integrated water quality conditions of tap water supplied to each building or a particular line of the corresponding building, tap water usage for each household, and the like may be automatically measured and recognized remotely and big data related to the water quality and usage of tap water may be constructed.

Second, according to the present invention, there is an effect in which a processor of the analysis server utilizes AI and administrates usage of tap water (for example, estimating and responding to ruddy tap water or the like) on the basis of stored water quality data.

Third, according to the present invention, there is a useful effect of remedying an expected problem for each household on the basis of tap water usage stored in the analysis server.

Other effects of the present invention will be obviously apparent to and understood by experts or researchers in the art through the detailed description or during a process of executing the present invention.

Although the present invention has been described with reference to specific matter such as detailed elements and the like, limited embodiments, and the drawings, these are provided only to aid in the overall understanding of the present invention. Accordingly, the present invention is not limited to the embodiments and one of ordinary skill in the art to which the present invention pertains may make a variety of changes and modifications.

Accordingly, it should be noted that the concept of the present invention is not defined by the above-described embodiments and the following claims and equivalents thereof belong to the scope of the concept of the present invention.

What is claimed is:

1. A method of measuring water quality of tap water while a water metering apparatus which receives power from a regular power supply receives a large amount of tap water supplied from a distribution pipe and transfers a certain amount of tap water to each household through a water supply pipe and a plurality of coupling members configured to connect such water supply pipes are present on the distribution pipe, the method comprising:
(a) obtaining, by the water metering apparatus, water quality data with respect to the supplied large amount of tap water using a water quality sensor installed on a particular coupling member among the plurality of coupling members; and
(b) transmitting, by the water metering apparatus, the water quality data with respect to the large amount of tap water, which is obtained from the water quality sensor, to an analysis server.

2. The method of claim 1, wherein the distribution pipe receives the large amount of tap water supplied from a water supply pipeline,
wherein the particular coupling member provided on the distribution pipe comprises a first particular coupling member, among the plurality of coupling members, which is present at a position closest to the water supply pipeline, and
wherein the first particular coupling member is coupled to a first water quality sensor in place of a first particular water supply pipe, and a sensing part of the first water quality sensor is located inside the distribution pipe and obtains first water quality data with respect to the large amount of tap water.

3. The method of claim 2, wherein, while a coupling member, among the plurality of coupling members, which is present at a position closest to the water supply pipeline next to the first particular coupling member, is referred to as a second particular coupling member and the second particular coupling member is included in the particular coupling member, the second particular coupling member is coupled to a second water quality sensor configured to measure another element different from that of the first water quality sensor in place of a second particular water supply pipe and a sensing part of the second water quality sensor is located inside the distribution pipe and obtains second water quality data with respect to the large amount of tap water, and
wherein the water supply pipes are connected to the remaining coupling members excluding the first particular coupling member and the second particular coupling member.

4. The method of claim 1, wherein the water quality data with respect to the large amount of tap water which is obtained using the water quality sensor comprises one or more pieces of data of pH, turbidity, a residual chlorine concentration, electrical conductivity, and a water temperature, and
wherein the electrical conductivity of tap water is calculated, at a reference temperature of 25 degrees, using the following equation:
$${C}_{25} = \frac{{C}_{t}}{1 + α ( t - 25 )} , \qquad Equation )$$
here, C₂₅ denotes an electrical conductivity value at 25 degrees, Cₜ denotes an electrical conductivity value at t degrees, and a corresponds to a linear temperature coefficient.

5. The method of claim 1, wherein the water quality sensor comprises at least one of a pH detection sensor configured to measure a hydrogen ion concentration quotient (pH) which is the activity of hydrogen ions present in tap water, a turbidity detection sensor configured to optically measure a light-scattering degree caused by floating matter by allowing light to be incident onto tap water, a residual chlorine concentration detection sensor configured to measure a concentration of chlorine remaining after disinfection to eliminate pathogenic bacteria in tap water, an electrical conductivity detection sensor, and a water temperature detection sensor.

6. The method of claim 1, wherein, while the water supply pipe is connected to each of the remaining coupling members of the plurality of coupling members excluding the particular coupling member and a plurality of water meters are each installed on one of the plurality of water supply pipes, the water metering apparatus obtains metering data with respect to certain amounts of tap water transferred to households using the plurality of water meters installed on the remaining coupling members and transmits a plurality of metering data obtained from the plurality of water meters to the analysis server.

7. The method of claim 1, wherein the water metering apparatus transmits the water quality data and the plurality of metering data to the analysis server using one communication module through long distance communication.

8. An apparatus for measuring water quality of tap water while a water metering apparatus which receives power from a regular power supply receives a large amount of tap water supplied from a distribution pipe and transfers a certain amount of tap water to each household through a water supply pipe and a plurality of coupling members configured to connect such water supply pipes are present on the distribution pipe, the apparatus comprising:
a water quality sensor installed on a particular coupling member among the plurality of coupling members;
a communication module configured to transmit water quality data to an analysis server; and
a control device configured to obtain water quality data with respect to the large amount of tap water through the water sensor and to transmit the water quality data with respect to the large amount of tap water, which is obtained from the water quality sensor, to an analysis server.
